(19) United States
(12) Patent Application Publication
(10) Pub. No.: US 2004/0106829 A1
(43) Pub. Date: Jun. 3, 2004

(54) Process for the synthesis of modafinil

(75) Inventors: Mirco Fornaroli, Cameri (IT); Francesco Velardi, Cameri (IT); Corrado Colli, Cameri (IT); Roberto Baima, Cameri (IT)

Correspondence Address: YOUNG & THOMPSON, 745 SOUTH 23RD STREET 2ND FLOOR, ARLINGTON, VA, 22202

(73) Assignee: PROCOS S.P.A., Cameri (IT)

(21) Appl. No.: 10/703,545

(22) Filed: Nov. 10, 2003

(30) Foreign Application Priority Data
Nov. 12, 2002 (IT) MI2002A002390

(51) Int. Cl. C07C 315/00

(52) U.S. Cl. 564/162

(57) ABSTRACT

The present invention relates to a process for the preparation of 2-(benzhydrylthio)acetamide (II), key intermediate for the synthesis of modafinil, by reaction of benzhydryl chloride with thiourea and chloroacetamide.

### FIELD OF THE INVENTION

The present invention relates to a process for the preparation of the intermediate 2-(benzhydrylthio)acetamide (II)

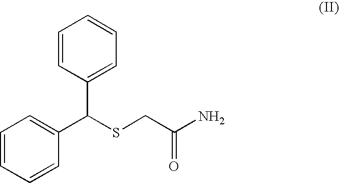

and to a process for the preparation of modafinil (2-[(diphenylmethyl)sulfinyl]acetamide) (I) from said intermediate (II).

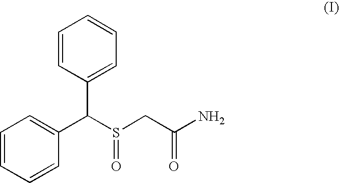

### TECHNOLOGICAL BACKGROUND

Modafinil acts on the central nervous system and is approved by the FDA for the treatment of narcolepsy. It is usually synthesized by oxidation of 2-(benzhydrylthio)acetamide, which in turn can be prepared in various ways. Some of these methods are shown in the following scheme.

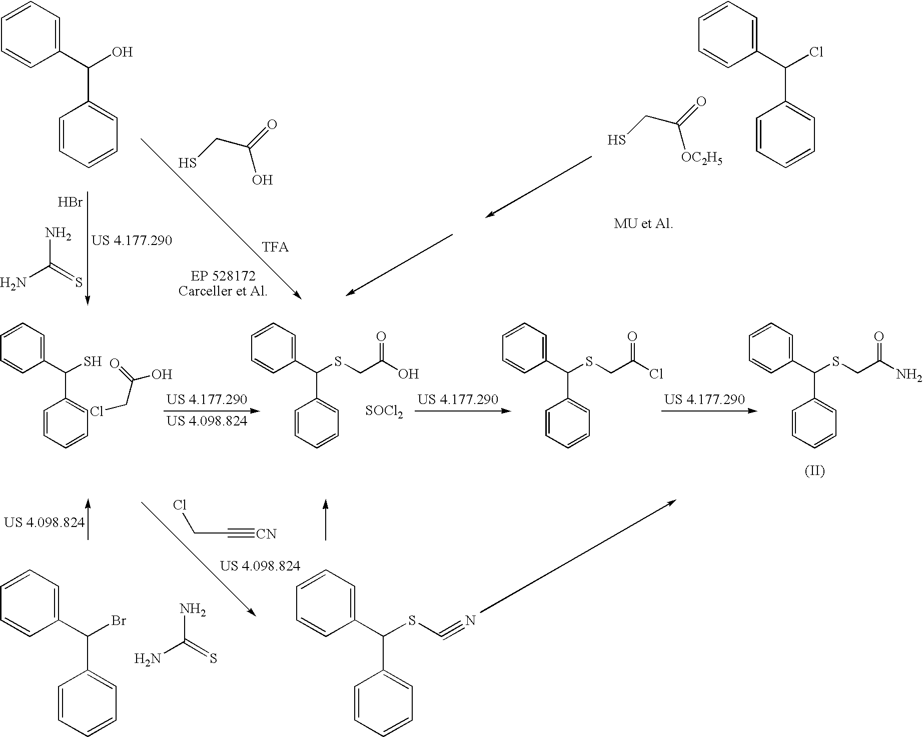

In U.S. Pat. No. 4,177,290 (example 1a) 2-(benzhydrylthio)acetic acid is prepared by first reacting benzhydrol with thiourea and 48% hydrobromic acid, then adding sodium hydroxide and chloroacetic acid. The reaction of 2-(benzhydrylthio)acetic acid (example 1) with thionyl chloride in benzene affords 2-(benzhydrylthio)acetil chloride, which is treated with ammonia to give 2-(benzhydrylthio)acetamide.

In U.S. Pat. No. 4,098,824 bromodiphenylmethane is reacted with thiourea to give diphenylmethanthiol, which is then reacted with chloroacetic acid to give 2-(benzhydrylthio)acetic acid (example 1b) or with chloroacetonitrile (example 2a) to give 2-(benzhydrylthio)acetonitrile. Both compounds can be transformed into 2-(benzhydrylthio)acetamide by known methods.

In EP 528172 and in Carceller et al. (Journal of Medicinal Chemistry 2984 (1993), p. 2988, scheme III, product 28A) benzhydrol is reacted with mercaptoacetic acid in trifluoroacetic acid to give 2-(benzhydrylthio)acetic acid, which is then converted to 2-(benzhydrylthio)acetamide.

In Mu et Al. (Zhongguo Yaowu Huaxue Zazhi (1999), 9(2), 132-134 (CA131:299268) chlorodiphenylmethane is reacted with 2-ethyl mercaptoacetate to give ethyl 2-(benzhydrylthio)acetate, from which compound (II) is subsequently obtained.

It will be appreciated that the above described methods require the recovery of different intermediates and the use of costly and/or toxic reagents. There is therefore the need to develop a method for the preparation of modafinil devoid of the aforementioned drawbacks.

### DESCRIPTION OF THE INVENTION

It has now been found that is possible to prepare 2-(benzhydrylthio)acetamide (II) from chlorodiphenylmethane without recovery of any of the intermediates and avoiding the use of toxic or dangerous reagents such as hydrobromic acid, trifluoroacetic acid or benzene.

The present invention relates to a “one pot” process for the preparation of 2-(benzhydrylthio)acetamide (II)

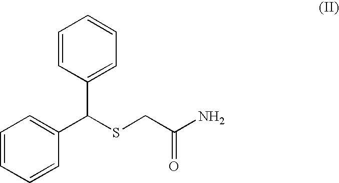

comprising the following steps:

a) reaction between benzhydryl chloride and thiourea in an aqueous medium in the presence of potassium iodide to give an isothiouronium salt of formula (III), wherein X is Cl, Br or I

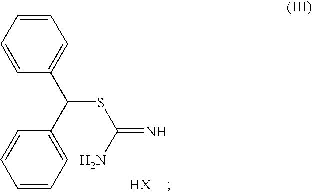

b) treatment of the isothiouronium salt (III) with an organic or inorganic base;

c) reaction of the compound from step b) with chloroacetamide in the presence of an organic base.

A further object of the present invention is a process for the preparation of modafinil (I) further comprising:

d) recovery of 2-(benzhydrylthio)acetamide by crystallization;

e) oxidation of 2-(benzhydrylthio)acetamide to give modafinil (I).

Step a) is typically carried out by adding benzhydryl chloride to an aqueous solution of thiourea and potassium iodide kept at a temperature from 60 to 80° C., preferably at 70° C., and then refluxing the mixture for 30 to 90 min.; during this period the thiouronium salt precipitates. The mixture is allowed to cool to room temperature and the hydrolysis of the salt (step b) is directly carried out by addition of water and a base, preferably a sodium hydroxide 30% aqueous solution or by addition of TEA.

Step c) is typically carried out by heating the mixture as directly obtained from the previous step to a temperature from 60 to 80° C., preferably to 70° C., and adding first the organic base, preferably triethylamine, then, over 30 to 90 minutes, chloroacetamide dissolved in water or dimethylsulfoxide. The mixture is refluxed for 30 to 90 minutes, until completion of the reaction.

Step d) can be carried out in two different ways. In a first embodiment of the invention the reaction mixture is cooled to 80° C. and crystallization is triggered by addition of 2-(benzhydrylthioacetamide) crystals.

In a second embodiment of the invention, the product is extracted in toluene and then crystallized from the same solvent. In more detail, the reaction mixture from step c) is cooled to a temperature from 70 to 90° C., preferably to 80° C.; upon addition of toluene, the formation of two phases is observed. The organic phase is separated, washed with hot water (70-90° C.) and concentrated by distilling off an aliquot of toluene at atmospheric pressure. Crystallization is achieved in 1-12 hours by cooling to room temperature, thereafter the product is filtered and washed first with toluene and then with hexane.

Compound (II) can be further recrystallized from toluene or ethyl acetate.

Step e) can be carried out by conventional methods for oxidizing sulfur to sulfoxide, preferably by treatment with acetic acid and hydrogen peroxide.

It will be appreciated from the foregoing that the process of the invention allows to prepare 2-(benzhydrylthio)acetamide and modafinil in a particularly advantageous way from the industrial standpoint, as steps a)-d) are carried out without recovering any of the intermediates, thus avoiding yield decrease and toxicity problems due to the work-up of the reaction mixtures and to the use of highly toxic or dangerous reagents or solvents.

The following examples illustrate the invention in more detail.

### EXAMPLE 1

### Synthesis of 2-(benzhydrylthio)acetamide

Thiourea (20.0 g) is loaded into a 500 ml round-bottom flask and water (60 ml) and potassium iodide are added (0.7 g). The resulting suspension is heated to 70° C. (at 35-40° C. complete dissolution is observed) and benzhydryl chloride (44.0 g) is added in 30 minutes. After the addition of the first one-quarter aliquot the mixture first becomes clear, then the product precipitates with exothermic reaction and heating (95° C.) is continued for 90 minutes.

The mixture, which is not easily stirrable, is cooled to 15-20° C., thereafter water (45 ml) is added, followed by 30% sodium hydroxide (29.0 g, i.e. the amount necessary to hydrolyse the isothiouronium salt) at a temperature of 24° C. The mixture is heated to 70° C. (at 50-60° C. the compound dissolves, and separation of a gluey but stirrable phase is observed) and triethylamine (24.1 g) is added (the gluey phase becomes an oil). A hot solution of chloroacetamide (22.0 g) in water (66 ml) is added in about 1 hour. An oil separates from the mixture and at the end of the addition compound (II) solidifies.

Refluxing is continued for 30 minutes, until all the solid becomes an oil again. Heating is stopped and at 80° C. crystallization is triggered with 2-(benzhydrylthio)acetamide crystals, thereafter stirring is continued overnight. The following day the mixture is cooled to 15° C., and the product is filtered after 1 hour and washed with water. Wet product: 64 g (HPLC: 81.4%).

The product is dissolved in boiling toluene, distilling off the amount of solvent necessary to remove the water. Decolorizing carbon (0.5 g) is added, then the mixture is hot filtered. Crystallisation is accomplished by slow cooling, then the product is filtered at 0-5° C. and dried at 40° C. under reduced pressure. Dry product: 45.6 g (HPLC: 99.3%).

### EXAMPLE 2

Thiourea (20.0 g) is loaded into a 500 ml round-bottom flask and water (60 ml) and potassium iodide (0.7 g) are added. The resulting suspension is on heated to 70° C. (at 35-40° C. complete dissolution is observed) and benzhydryl chloride (44.0 g) is added in 30 minutes. After the addition of the first one-quarter aliquot the mixture first becomes clear, then the product precipitates with exothermic reaction and heating (70-75° C.) is continued for 60 minutes.

The mixture, which is not easily stirrable, is cooled to 15-20° C., thereafter water (45 ml) is added, followed by 30% sodium hydroxide (29.0 g, i.e. the amount necessary to hydrolyse the isothiouronium salt), at a temperature of 24° C. The mixture is heated to 70° C. (at 50-60° C. the product dissolves, and the separation of a gluey but stirrable phase is observed) and 24.1 g of triethylamine is added (the gluey phase becomes an oil). The temperature is raised to 80° C. and a solution of chloroacetamide (22.0 g) in dimethylsulfoxide (33 ml) is added over approximately 1 hour; an oil separates from the mixture. The temperature is kept at 80° C. for 60 minutes and then toluene (250 ml) is added. The phases are separated at 80° C. and the organic one is washed with 150 ml of hot water. The phases are separated and the aqueous one is re-extracted with toluene (80 ml), then separated at 80° C. The organic phase from the first extraction is concentrated by distilling off 150 ml of toluene at atmospheric pressure, to obtain a clear solution, which is allowed to cool to room temperature overnight. After further cooling to 0-5° C. for 30 minutes, the crystallized product is filtered and washed, first with 15 ml of toluene at 0° C. and then with 50 ml of hexane at 0° C. Wet product: 54.1 g. After drying at 50° C. under reduced pressure for 4 hours and 30 minutes 42.3 g of dry product is obtained (HPLC: 97.9%).

### EXAMPLE 3

The same procedure as that of example 1 or 2 is followed until convertion to 2-(benzhydrylthio)acetamide is complete (solidification of the product in the mixture).

The mixture is then refluxed for 30 minutes, until the solid becomes an oil again. Heating is stopped and at 80° C. toluene is added (250 ml). The phases are separated at 80° C. and the organic one is hot washed with 150 ml of hot water, then concentrated by distilling off 150 ml of toluene at atmospheric pressure. Diatomaceous earth is added, the resulting mixture is hot filtered and allowed to crystallize overnight by gradually cooling. The following day, after cooling to 0-5° C., the product is filtered, washed with toluene and dried at 40° C. under reduced pressure. Yield: 47.3 g (wet: 57.3 g) (HPLC: 94.1%).

### EXAMPLE 4

The same procedure as that of example 1 or 2 is followed until complete hydrolysis of the isothiouronium salt and addition of triethylamine, then a solution of chloroacetamide (22.0 g) in dimethylsulfoxide (33 ml) is added in about 1 hour. A solution containing a suspended oil is obtained at the end of the addition. This solution is refluxed for 30 minutes, then heating is stopped and, when the temperature reaches 80° C., 250 ml of toluene is added, and marked and rapid separation of the phases is observed. The organic phase is washed with hot water (150 ml). The phases are separated at 80° C. and the organic phase from the first extraction is concentrated by distilling off 150 ml of toluene at atmospheric pressure, to obtain a clear solution.

Crystallization is achieved in 1 h and 30 minutes by cooling to room temperature. After cooling at 0-5° C. for 30 minutes the product is filtered and washed first with 15 ml of toluene (at 0° C.) and then with 50 ml of hexane (at 0° C.). The wet product (52.5 g) is dried at 50° C. under reduced pressure for 4 hours.

Dry product: 44 g (HPLC: 97.9%)

### EXAMPLE 5

The same procedure as that of example 1 or 2 is followed, but after addition of 45 ml of water to the mixture containing the isothiouronium salt, hydrolysis is accomplished by addition of 22 g of triethylamine at 25° C. The addition is slightly exothermic and precipitation of a white solid is observed. The mixture is then heated to 70° C. (the product does not dissolve completely) and 24.1 g of triethylamine is added. The solid agglomerates and at the end of the addition becomes gluey. After stirring for 15 minutes a suspended oil is obtained, thereafter a solution of chloroacetamide (22.0 g) in dimethylsulfoxide (33 ml) at room temperature is added over about 1 hour, the addition being slightly exothermic and a suspended oil is obtained.

The mixture is heated to 96°-100° C. for 30 minutes, then heating is stopped and when the temperature has decreased to 80° C. toluene is added (250 ml). The phases are separated at 80° C. and the organic one is washed with hot water (150 ml). The phases are separated and the aqueous one is re-extracted with toluene (80 ml), then separated at 80° C. The organic phase from the first extraction is concentrated by distilling off 150 ml of toluene at atmospheric pressure, to obtain a clear solution, which is allowed to cool to room temperature in 1 hour and 30 minutes. After further cooling to 0-5° C. for 30 minutes, the crystallized product is filtered and washed, first with 15 ml of toluene at 0° C. and then with 50 ml of hexane at 0° C. Wet product: 48.6 g. The wet product is dried at 50° C. under reduced pressure for 4 hours and 30 minutes. Dry product: 42.3 g (HPLC: 94.9%).

### EXAMPLE 6

Anhydrous crude (HPLC 80.2%) 2-(benzhydrylthio)acetamide (40.0 g) and ethyl acetate (160 ml) are loaded into a 500 ml round-bottom flask and the resulting mixture is stirred and refluxed for 10 minutes. As soon as the solution becomes slightly turbid, diatomaceous earth (0.5 g) is added and hot filtered into a conic flask with a pleated filter. The product is allowed to crystallize by cooling to room temperature under stirring, kept at 0°-5° C. for 15 minutes, then filtered and washed with ethyl acetate (20 ml) at 0° C. Wet product: 38.9 g. After drying at 50° C. under reduced pressure overnight 27.0 g (HPLC 95.6%) of dry product is obtained.

### EXAMPLE 7

### Synthesis of Modafinil

2-(Benzhydrylthio)acetamide obtained according to Example 4 (30 g-HPLC 97.9%) and acetic acid (120 ml) are loaded into a 1000 ml round-bottom flask. The mixture is heated to 40° C., slowly added with 35% hydrogen peroxide (11.7 g) and reacted at 40° C. for about 6 hours. The mixture is then cooled to 30° C., added with water (900 ml) and further cooled to 15° C. The product is filtered and washed with water. The wet product (49.4 g) thus obtained (HPLC: 95.73%, 0.083% (sulfone)) is dried at 50° C. under reduced pressure. Yield: 29.7 g.

Crude modafinil is recrystallized from methanol (193 ml), by heating under reflux and cooling then to 15° C. The precipitate is filtered, washed with cold methanol and dried at 50° C. under reduced pressure. Yield: 23.4 g (HPLC: 99.9%, 0.01% (sulfone)).

1. “One pot” process for the preparation of 2-(benzhydrylthio)acetamide (II), 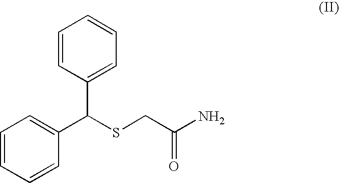
comprising the following steps:
a) reaction of benzhydryl chloride and thiourea in an aqueous medium in the presence of potassium iodide to give an isothiouronium salt of formula (III) wherein X is Cl, Br or I 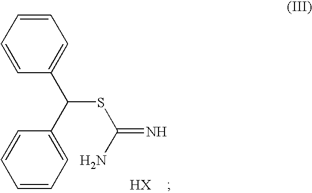
b) treatment of the isothiouronium salt (III) with an organic or inorganic base;
c) reaction of the compound from step b) with chloroacetamide in the presence of an organic base:

2. Process for the preparation of modafinil (I) 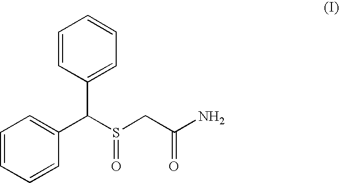
comprising the following steps:
a) reaction of benzhydryl chloride and thiourea in an aqueous medium in the presence of potassium iodide to give an isothiouronium salt of formula (III) wherein X is Cl, Br or I 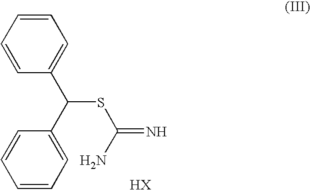
b) treatment of the isothiouronium salt (III) with an organic or inorganic base;
c) reaction of the compound from step b) with chloroacetamide in the presence of an organic base to give 2-(benzhydrylthio)acetamide (II) 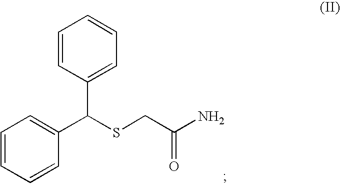
d) recovery of 2-(benzhydrylthio)acetamide by crystallization;
e) oxidation of 2-(benzhydrylthio)acetamide to modafinil (I).

3. Process as claimed in claim 1 or 2 characterized in that the reactions of the steps a) and c) are carried out at a temperature ranging from 60 to 80° C.

4. Process according to any one of claims 1-3 characterized in that the organic base used in the step b) and c) is triethylamine.

5. Process according to any one of claims 1-3 characterized in that the inorganic base used in the step b) is 30% sodium hydroxide.
